(19) United States
(12) Patent Application Publication
(10) Pub. No.: US 2024/0219308 A1
(43) Pub. Date: Jul. 4, 2024

(54) COLORIMETRIC DETECTION OF TARGET MATERIAL BASED ON HYDROGEL PARTICLE

(71) Applicant: Korea University Research and Business Foundation, Seoul (KR)

(72) Inventors: Ki Wan BONG, Seoul (KR); Yoohno ROH, Seoul (KR); Seok Joon MUN, Seoul (KR); Hyun Jee LEE, Seoul (KR); Ju Yeon KIM, Seoul (KR)

(73) Assignee: Korea University Research and Business Foundation, Seoul (KR)

(21) Appl. No.: 17/769,601

(22) PCT Filed: Oct. 15, 2020
(86) PCT No.: PCT/KR2020/014110

§ 371 Date: Apr. 15, 2022

(30) Foreign Application Priority Data
Oct. 18, 2019 (KR) 10-2019-0130215

(51) Int. Cl. G01N 21/78; G01N 33/543; G01N 33/58

(52) U.S. Cl. CPC G01N 21/78; G01N 33/54313; G01N 33/581

(57) ABSTRACT

The present invention relates to a colorimetric detection of a target material based on a hydrogel particle. A colorimetric detection of a target material based on a hydrogel particle according to the embodiment of the present invention comprises the steps of: reacting a sample containing the target material with the hydrogel particle, which is loaded on the inside with a probe that specifically binds to the target material; and accumulating and amplifying an insoluble colorimetric material in the hydrogel particle so as to label the target material bound to the probe. Here, the hydrogel particle is made of a polymer network, the probe is loaded by binding to the polymer network, and the insoluble colorimetric material is fixed to the polymer network.

### TECHNICAL FIELD

The present invention relates to a method for colorimetric detection of target analytes based on hydrogel particles, and more specifically to a method for detecting target analytes by introducing colorimetric reactions in hydrogel particles.

### BACKGROUND ART

Encoded hydrogel particles have attracted much attention in fields, including diagnostic medicine, drug screening, and molecular biochemistry, where high detection performance for target biomolecules is required because they enable multiplexed detection of target biomolecules with high sensitivity and specificity. Target analytes bound to encoded hydrogel particles may be labeled with fluorescent materials. However, the use of expensive systems such as light sources, microscopes, and cameras is essential for fluorescence analysis, making it difficult to commonly use fluorescent materials in places where fluorescence analysis systems are not available. When exposed to light during fluorescence labeling or during washing after fluorescence labeling, fluorescent materials may lose their fluorescence properties due to photobleaching. Further, non-specific binding of fluorescent materials to supports such as substrates or membranes may generate false-positive signals, affecting the reliability of quantitative analysis. Fluorescent materials may be used for qualitative analysis as well as quantitative analysis. Quantitative analysis using fluorescent materials also requires the same expensive systems as used for quantitative analysis to determine the binding of target analytes. Further, quantitative analysis should be done in limited spaces such as darkrooms to prevent photobleaching. Due to these problems, fluorescence analysis should be performed only in centralized laboratories and is difficult to extend to point-of-care tests (POCTs) whose majority is accounted for by qualitative analysis.

Thus, there is a need to develop a new technology that can analyze biomolecules without using expensive systems or space limitation with high sensitivity comparable to conventional fluorescence assays.

### DETAILED DESCRIPTION OF THE INVENTION

### Problems to be Solved by the Invention

The present invention has been made in an effort to solve the problems of the prior art and one aspect of the present invention is to provide a method for colorimetric detection of target analytes based on hydrogel particles in which probes are loaded into hydrogel particles, the target analytes specifically bind to the probes, and an insoluble colorimetric material is accumulated and amplified in the hydrogel particles.

### Means for Solving the Problems

A method for colorimetric detection of target analytes based on hydrogel particles according to an embodiment of the present invention includes (a) reacting a sample containing target analytes with hydrogel particles loaded with probes specifically binding to the target analytes and (b) accumulating and amplifying an insoluble colorimetric material in the hydrogel particles to label the target analytes bound to the probes wherein the hydrogel particles form a polymer network, the probes are bound to and loaded into the polymer network, and the insoluble colorimetric material is fixed to the polymer network.

The hydrogel particles may be geometrically shaped and encoded to identify the probes.

The probes may be loaded during or after synthesis of the hydrogel particles.

Each of the probes loaded after synthesis of the hydrogel particles may include a capture portion specifically binding to the corresponding target analyte and a functional group connected to the capture portion and bonded to an unreacted end in the form of a carbon-carbon double bond connected to the polymer network.

The probes may be compounds, oligonucleotides, oligosaccharides, proteins, antibodies, peptides or aptamers that specifically bind to the target analytes. The functional groups may be selected from the group consisting of thiol (—SH) and amine groups (—NH₂).

Step (b) may include conjugating an enzyme to the target analytes bound to the probes and adding a substrate reacting with the enzyme to produce the insoluble colorimetric material.

The conjugation of the enzyme may include adding secondary binding materials specifically binding to the target analytes and adding the enzyme for binding to the secondary binding materials.

The secondary binding materials may be compounds, oligonucleotides, oligosaccharides, proteins, antibodies, peptides or aptamers that specifically bind to the target analytes.

The enzyme may be selected from alkaline phosphatase (ALP), 0-galactosidase, peroxidase, luciferase, cytochrome P450, and combinations thereof.

The substrate may be selected from bromochloroindolyl phosphate (BCIP)/nitroblue tetrazolium (NBT), naphthol-AS-B1-phosphate, p-nitrophenyl phosphate (PNPP), enhanced chemifluorescence (ECF), 4-chloronaphthol, 3,3′-diaminobenzidine (DAB), 3-amino-9-ethylcarbazole (AEC), 3,3′,5,5′-tetramethylbenzidine (TMB), 4-chloronaphthol, 3,3′-diaminobenzidine (DAB), 3-amino-9-ethylcarbazole (AEC), 6-chloro-3-indolyl-o-D-galactopyranoside (Red-gal), and combinations thereof.

The target analytes may include one or more materials selected from the group consisting of DNA, RNA, proteins, exosomes, and viruses.

The features and advantages of the present invention will become apparent from the following description with reference to the accompanying drawings.

Prior to the detailed description of the invention, it should be understood that the terms and words used in the specification and the claims are not to be construed as having common and dictionary meanings but are construed as having meanings and concepts corresponding to the technical spirit of the present invention in view of the principle that the inventor can define properly the concept of the terms and words in order to describe his/her invention with the best method.

### Effects of the Invention

According to the present invention, an insoluble colorimetric material is accumulated and amplified in hydrogel particles instead of labeling target analytes (for example, nucleic acids and proteins) bound to the inside of hydrogel particles with a fluorescent material, to obtain results comparable to the detection sensitivity and specificity achieved using a fluorescent material even without using expensive analysis systems or a separate space.

According to the present invention, a colorimetric material labeling target analytes in particles can be detected only with the naked eye or in a bright field image, enabling quantification of the target analytes only with simple systems, including a USB microscope and a smartphone, without the need for expensive systems. In addition, the colorimetric material is accumulated and amplified in the hydrogel particles, achieving performance comparable to the detection sensitivity and specificity of fluorescence assays which could not been achieved by existing colorimetric reactions. The accumulation and amplification of the colorimetric material enables quantitative analysis of target analytes with high reliability even in a place rather than a limited space such as a darkroom because no photobleaching occurs. Therefore, the present invention can be widely utilized in the field of point-of-care testing (POCT) outside centralized laboratories as well as for quantitative analysis of target analytes such as nucleic acids and proteins.

### BRIEF DESCRIPTION OF THE DRAWINGS

FIG. 1 is a diagram schematically illustrating a process for synthesizing hydrogel particles used in a method for colorimetric detection of target analytes based on hydrogel particles according to an embodiment of the present invention.

FIG. 2 shows plan views of various shapes of hydrogel particles synthesized by the process of FIG. 1.

FIG. 3 is a diagram illustrating a process for binding probes to unreacted ends remaining in hydrogel particles synthesized by the process of FIG. 1.

FIGS. 4 and 5 are diagrams illustrating a process in which after detection of target analytes in a method for colorimetric detection of target analytes based on hydrogel particles according to an embodiment of the present invention, a colorimetric material is made insoluble through an enzyme-substrate reaction in hydrogel particles by a labeled enzyme and is accumulated and amplified.

FIG. 6 graphically shows the results of single detection of three types of preeclampsia-related proteins by a colorimetric detection method in Experimental Example 3.3.

FIG. 7 shows images revealing the results of single detection of three types of preeclampsia-related proteins by a colorimetric detection method in Experimental Example 3.3.

FIG. 8 shows images revealing the results of multiplexed detection of three types of preeclampsia-related proteins by a colorimetric detection method in Experimental Example 3.4.

FIG. 9 graphically shows the results of multiplexed detection of three types of preeclampsia-related proteins by a colorimetric detection method in Experimental Example 3.4.

FIG. 10 compares the results of detection of proteins spiked in plasma by a colorimetric detection method in Experimental Example 3.5 with the results of ELISA.

FIG. 11 is diagram schematically illustrating a process in which a colorimetric detection method is applied to plasma extracted from a real preeclampsia patient and a USB microscope and a smartphone are used for analysis in Experimental Example 3.5.

FIG. 12 shows the results of multiplexed detection of two types of proteins in plasma samples extracted from real preeclampsia patients and healthy subjects by a colorimetric detection method in Experimental Example 3.5.

FIG. 13 shows the results of single detection of nucleic acids by a colorimetric detection method in Experimental Example 3.6.

FIG. 14 shows the results of multiplexed detection of nucleic acids by a colorimetric detection method in Experimental Example 3.6.

### BEST MODE FOR CARRYING OUT THE INVENTION

The objects, specific advantages, and novel features of the present invention will become more apparent from the following detailed description and preferred embodiments, examples of which are illustrated in the accompanying drawings. A detailed description of well-known technologies is avoided lest it should obscure the subject matter of the invention.

Preferred embodiments of the present invention will now be described in detail with reference to the accompanying drawings.

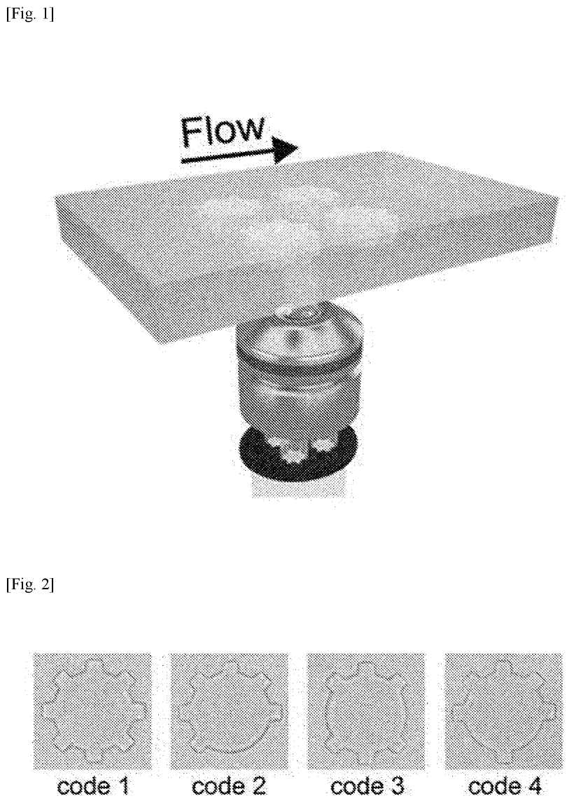

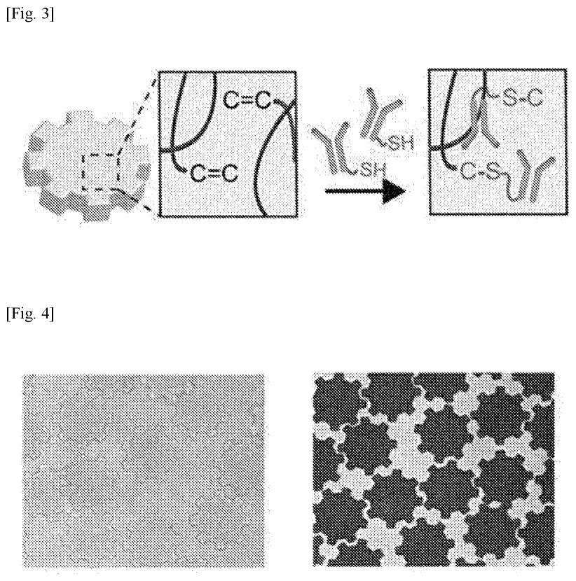

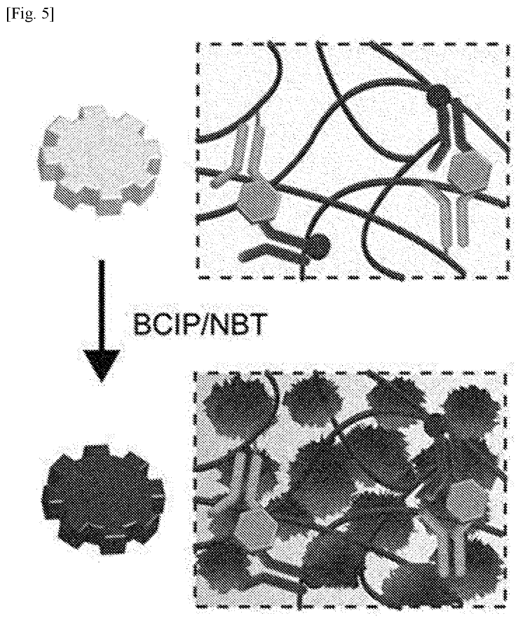

FIG. 1 is a diagram schematically illustrating a process for synthesizing hydrogel particles used in a method for colorimetric detection of target analytes based on hydrogel particles according to an embodiment of the present invention. FIG. 2 shows plan views of various shapes of hydrogel particles synthesized by the process of FIG. 1. FIG. 3 is a diagram illustrating a process for binding probes to unreacted ends remaining in hydrogel particles synthesized by the process of FIG. 1. FIGS. 4 and 5 are diagrams illustrating a process in which after detection of target analytes in a method for colorimetric detection of target analytes based on hydrogel particles according to an embodiment of the present invention, a colorimetric material is made insoluble through an enzyme-substrate reaction in hydrogel particles by a labeled enzyme and is accumulated and amplified.

As shown in FIGS. 1 to 5, a method for colorimetric detection of target analytes based on hydrogel particles according to an embodiment of the present invention includes: reacting a sample containing target analytes with hydrogel particles loaded with probes specifically binding to the target analytes; and accumulating and amplifying an insoluble colorimetric material in the hydrogel particles to label the target analytes bound to the probes. The hydrogel particles form a polymer network, the probes are bound to and loaded into the polymer network, and the insoluble colorimetric material is fixed to the polymer network.

The method of the present invention uses colorimetric reactions introduced in hydrogel particles to detect target analytes. Conventional fluorescence assays using fluorescent materials to detect target analytes require the use of expensive systems such as light sources, microscopes, and cameras for fluorescence analysis. When exposed to light, fluorescent materials may be photobleached and may non-specifically bind to supports to generate false-positive signals, affecting the reliability of quantitative analysis. Quantitative analysis using fluorescent materials also requires expensive systems and should be done in limited spaces such as darkrooms to prevent photobleaching. Due to these problems, fluorescence analysis should be performed only in centralized laboratories and is difficult to extend to point-of-care tests (POCTs) whose majority is accounted for by qualitative analysis. The present invention provides a solution to the problems of conventional fluorescence assays.

Specifically, the method of the present invention includes reacting a sample with probe-loaded hydrogel particles and accumulating and amplifying an insoluble colorimetric material.

First, a sample is allowed to react with probe-loaded hydrogel particles. The probe-loaded hydrogel particles form a polymer network and have a plurality of pores extending from the outside to the inside. The probes are bound to and loaded into the polymer network.

In the probe-loaded hydrogel particles, the probes bind to target analytes. This reaction is similar to that in solution and can proceed three-dimensionally, enabling detection of biomolecules with high specificity and sensitivity in a wide dynamic range. The probes specifically bind to target analytes. Specifically, when a sample containing target analytes is mixed with probe-loaded hydrogel particles, the target analytes penetrate into the hydrogel particles through pores of the hydrogel particles and specifically bind to the probes loaded into the hydrogel particles.

Referring to FIG. 2, the probe-loaded hydrogel particles can be encoded. Here, the hydrogel particles may be geometrically shaped and encoded to identify the loaded probes. The hydrogel particles may be loaded with different probes. In this case, the hydrogel particles have different shapes as codes assigned to identify the probes. Accordingly, the hydrogel particles and the loaded probes can be distinguished depending on the codes imparted to the particles, enabling simultaneous detection of multiple target analytes. In FIG. 2, the hydrogel particles are encoded with up to eight rectangular gear-shaped projections. Alternatively, the hydrogel particles may be encoded with various geometric shapes.

The encoded probe-loaded hydrogel particles can be synthesized by flow lithography. Referring to FIG. 1, ultraviolet light is irradiated onto a flowing precursor fluid to synthesize particles by flow lithography. Ultraviolet light passing through a photomask causes polymerization of the precursor to synthesize particles having the same shape as the photomask. The fluid forms a laminar flow in a microchannel and can be structured in multiple parallel flows without mixing, enabling the synthesis of multifunctional asymmetric particles upon UV irradiation.

The precursor fluid may include a photocurable monomer having a carbon-carbon double bond (C═C) as a functional group and a photoinitiator. The photocurable monomer having a carbon-carbon double bond as a functional group may be, for example, methacrylate, maleimide, vinyl sulfone, acrylate or acrylamide. The precursor fluid may further include a porogen such as polyethylene glycol. Deionized water (DI water) may be used as a solvent for dispersing the porogen. In the hydrogel particles synthesized using the precursor fluid, the photocurable monomer molecules are polymerized to form a polymer network structure. The highly reactive and biochemically unstable carbon-carbon double bonds of the monomer molecules are cross-linked to form a network during synthesis of the particles. The carbon-carbon double bonds are transformed into very less reactive single bonds after cross-linking. However, not all functional groups of the monomer molecules present in the precursor fluid are transformed during flow lithography and some of the carbon-carbon double bonds remain unreacted. Some of the unreacted carbon-carbon double bonds are bound to the network. That is, some of the functional groups of the monomer molecules are cross-linked in the network after reaction but some of the functional groups of the monomer molecules remain unreacted. The unreacted functional groups in the form of carbon-carbon double bonds connected to the network are defined as unreacted ends.

Since the unreacted ends are not removed even by rinsing after particle synthesis, they are present in the hydrogel particles synthesized by flow lithography, as shown in FIG. 3. The probes are bonded to the unreacted ends connected to the polymer network.

Each probe may include a capture portion specifically binding to a target analyte and a functional group connected to the capture portion and bonded to the unreacted end. The functional groups of the probes bonded to the unreacted ends in the form of carbon-carbon double bonds may be selected from the group consisting of thiol (—SH) and amine groups (—NH₂). The thiol-ene click reaction is used for the reaction between the carbon-carbon double bond and the thiol. The thiol-ene click reaction is very fast, reaches a yield of almost 100%, and produces no side reactions or by-products. The aza-Michael addition reaction can be utilized for the reaction between the carbon-carbon double bond and the amine.

These reactions may be radical reactions, catalytic reactions or spontaneous reactions. The capture portions of the probes may be cross-linked to the inside of the hydrogel particles. According to the radical reaction, the hydrogel particles are dispersed in a medium such as water or a solvent, capture portions (probes) containing functional groups capable of reacting with the unreacted ends through a radical reaction are added and dispersed, a photoinitiator or thermal initiator is added, and light (UV) or thermal energy is applied for a predetermined period of time to induce covalent bonds between the carbon-carbon double bonds and the functional groups. As a result, the probes are loaded in the hydrogel particles and the unreacted ends are removed. The catalytic reaction refers to a catalyst-mediated reaction. The catalytic reaction uses an organic catalyst instead of the initiator used in the radical reaction, to induce covalent bonds between the carbon-carbon double bonds and the functional groups. As a result, the probes are loaded and the unreacted ends are removed. The spontaneous reaction is suitable for loading probes sensitive to radicals or catalysts. According to the spontaneous reaction, the probes are cross-linked to the hydrogel particles through electrons moving in solution. The electrons of the buffer or polar solvent act as nucleophiles to form covalent bonds between the functional groups and the carbon-carbon double bonds.

However, the synthesis of the encoded hydrogel particles is not necessarily limited to flow lithography. Various processes, including replica molding, can be used to synthesize the encoded hydrogel particles. Replica molding is a process for synthesizing particles by loading a fluid into a micro-mold engraved with an intaglio micro-pattern and irradiating ultraviolet light onto the fluid. The shape of the particles is the same as the intaglio pattern engraved on the micro-mold.

The probes for the detection of target analytes may be loaded not only after, but also during synthesis of the hydrogel particles. The probes (capture portions) are materials substances capable of specifically binding to target analytes. For example, the probes (capture portions) may be compounds, oligonucleotides, oligosaccharides, proteins, antibodies, peptides or aptamers that specifically bind to target analytes. Particularly, antibodies as the probes (capture portions) specifically bind to target analytes through an antigen-antibody reaction. The target analytes are not particularly limited as long as they specifically bind to the probes (capture portions). The target analytes may include one or more materials selected from the group consisting of DNA, RNA, proteins, exosomes, and viruses.

Then, an insoluble colorimetric material is accumulated and amplified. The insoluble colorimetric material labels the target analytes bound to the probes to determine the binding of the target analytes. The insoluble colorimetric material aggregates locally in a hydrophilic environment and is fixed to the polymer network of the probe-loaded hydrogel particles. As a result, the insoluble colorimetric material does not flow out of the particles but is accumulated and amplified in the hydrogel particles. As shown in FIG. 4, the insoluble colorimetric material labeling the target analytes in the particles can be detected only with the naked eye or in a bright field image. Accordingly, the target analytes can be quantified only with simple systems, including a USB microscope and a smartphone, without the need for expensive systems. The accumulation and amplification of the colorimetric material in the hydrogel particles can achieve performance comparable to the detection sensitivity and specificity of fluorescence assays and enable quantitative analysis of target analytes with high reliability even in a place rather than a limited space such as a darkroom because no photobleaching occurs.

In one embodiment where the insoluble colorimetric material is accumulated and amplified in the hydrogel particles, an enzyme is conjugated to the target analytes bound to the probes and a substrate reacting with the enzyme is added to produce the insoluble colorimetric material. The enzyme may be selected from alkaline phosphatase (ALP), 0-galactosidase, peroxidase, luciferase, cytochrome P450, and combinations thereof. The substrate may be selected from bromochloroindolyl phosphate (BCIP)/nitroblue tetrazolium (NBT), naphthol-AS-BI-phosphate, p-nitrophenyl phosphate (PNPP), enhanced chemifluorescence (ECF), 4-chloronaphthol, 3,3′-diaminobenzidine (DAB), 3-amino-9-ethylcarbazole (AEC), 3,3′,5,5′-tetramethylbenzidine (TMB), 4-chloronaphthol, 3,3′-diaminobenzidine (DAB), 3-amino-9-ethylcarbazole (AEC), 6-chloro-3-indolyl-β-D-galactopyranoside (Red-gal), and combinations thereof.

For example, alkaline phosphatase (ALP) and 5-bromo-4-chloro-3-indolyl phosphate/nitroblue tetrazolium (BCIP/NBT) may be used as the enzyme and the substrate, respectively. In this case, the target analytes captured in the probe-loaded hydrogel particles are labeled with the enzyme ALP and the BCIP/NBT substrate solution is introduced into the hydrogel particles, resulting in accumulation and amplification of the insoluble colorimetric material through an enzyme-substrate reaction in the hydrogel particles. As a result, the hydrogel particles are colored dark purple (see FIG. 5). The target analytes may be labeled with the enzyme via secondary binding materials specifically binding to the target analytes bound to the probes. The secondary binding materials may be compounds, oligonucleotides, oligosaccharides, proteins, antibodies, peptides or aptamers that specifically bind to the target analytes. The secondary binding materials and the enzyme may be added sequentially to induce the binding between the secondary binding materials and the enzyme. Alternatively, the secondary binding materials polymerized with the enzyme may be added. In one embodiment, the secondary binding materials may be secondary antibodies specifically binding to the target analytes through an antigen-antibody reaction. In this embodiment, biotinylated secondary antibodies specifically bind to the target analytes captured by the probes, streptavidin-polymerized ALP (streptavidin-ALP) is added such that the streptavidin binds to the biotin, and BCIP/NBT is added, resulting in accumulation and amplification of the insoluble colorimetric material in the hydrogel particles.

Overall, according to the present invention, when the probe-loaded hydrogel particles are mixed with the sample and allowed to stand for detection reactions for a predetermined time, only materials specific to the probes loaded into the particles bind to the probes. After completion of the detection reactions, the binding between the probes and the target analytes needs to be identified to determine whether the target analytes are bound to the hydrogel particles. According to a conventional fluorescence assay, binding portions are labeled with fluorescent materials and the presence and intensity of fluorescence are analyzed with a fluorescence analyzer including a light source, a microscope, and a camera. In contrast, according to the present invention, the accumulation and amplification of the insoluble colorimetric material in the hydrogel particles can achieve performance comparable to the detection sensitivity and specificity of fluorescence assays while avoiding the problems of fluorescence assays.

### MODE FOR CARRYING OUT THE INVENTION

The present invention will be explained more specifically with reference to the following experimental examples.

### 1. Experimental Examples

### 1.1. Fabrication of Microfluidic Chip

A microfluidic chip for synthesizing hydrogel particles was designed using AutoCAD (Autodesk, CA, USA) and printed on a photomask film (Han&All technology, Korea). SU-8 25, a negative photoresist, was coated to a thickness of 52 μm on a silicon wafer, followed by photolithography to construct an SU-8 master mold. A mixture of PDMS (Corning, USA) and a curing agent in a weight ratio of 10:1 was poured into the SU-8 master mold and cured at 70° C. for 8 h. The cured PDMS was separated from the SU-8 master mold and cut into sections. Thereafter, an inlet and an outlet of a channel engraved on the section were drilled using 1.0 mm and 10.0 mm biopsy punches. A mixture of PDMS and a curing agent in a weight ratio of 10:1 was poured into a glass slide and partially cured at 70° C. for 25 min. The prepared section was attached to the surface of the glass slide and cured at 70° C. overnight to fabricate a microfluidic chip.

### 1.2. Stop Flow Lithography (SFL) Setup

Hydrogel particles were synthesized by stop flow lithography (see FIG. 1). For SFL, a system for UV and pressure control was constructed using a customized circuit board and the LabView (National Instruments, TX, USA) program. The microfluidic chip was placed on an inverted microscope (Zeiss, Germany) and a precursor was injected into the microfluidic chip through a pipette tip. Here, the precursor was injected by air whose pressure was controlled using a pressure regulator. A photomask engraved with various patterns was fixed to the field-stop of the microscope and an LED lamp was used as a source for curing the precursor. The UV intensity was maintained at 2200 mW cm².

### 1.3. Preparation of Antibody-Functionalized Hydrogel Particles

The precursor injected into the microfluidic chip was composed of 20% (v/v) polyethylene glycol diacrylate (PEG700DA, Sigma Aldrich, USA), 40% (v/v) polyethylene glycol 600 (PEG600, Sigma Aldrich) as a porogen, 35% (v/v) deionized water, and 5% (v/v) Darocur 1173 (Sigma Aldrich) as a photoinitiator. The precursor injected into the microfluidic chip was synthesized into hydrogel particles through continuous cycles of flow (400 ms), stop (200 ms), UV exposure (65 ms), and hold times (335 ms). Different photomasks were used to encode protein groups. The synthesized particles were rinsed 3 times in 1×PBST (phosphate buffer containing 0.005% Tween 20). Next, 12 μl of reconstituted capture antibody (12 μg μl⁻¹ for P1GF, 6 μg μl⁻¹ for sFIT-1, 6 μg μl⁻¹ for sEng) was reacted with 16.5 μl of the particles (˜75 per μl) and stirred with 1.5 μl of a dissimilar functional PEG linker (Thiol-PEG 2000-NHS) at 1500 rpm at 25° C. The resulting antibody-conjugated hydrogel microparticles were stored in 1×PBST at 4° C.

### 1.4. Protein Detection by Colorimetric Reactions

Protein detection reactions were carried out in volumes of 80 μl. In each detection reaction, 40 μl (˜50) of antibody-loaded hydrogel particles in 1×PBST were mixed with 2× target protein in FBS. The reaction was allowed to proceed at 1500 rpm and 25° C. for 2 h. After completion of the reaction, the reaction mixture was rinsed 3 times with 1×PBST and secondary antibody (15 ng μl⁻¹ for P1GF, 125 ng μl⁻¹ for sFIT-1, 12.5 ng μl⁻¹ for sEng) was added. The reaction was again carried out at 1500 rpm and 25° C. for 1 h.

After rinsing 3 times with 1×PBST, the hydrogel particles were labeled with an enzyme through streptavidin-AP in 1% BSA. Finally, 50 μl of BCIP/NBT solution was mixed with the particles in a microtube, followed by reaction at 25° C. for 7 min. After completion of the reaction, the reaction mixture was rinsed 3 times with DI water+tween 20 solution. A bright field image was taken through a USB microscope connected to a laptop (Insan commerce, Korea), a 3D printed stage, and a home-made light source system.

### 2. Comparative Example

### ELISA

A 96-well microplate was coated with 100 μl of capture antibody (4 μg μl⁻¹ for P1GF, 2 μg μl⁻¹ for sFIt-1, 2 μg μl⁻¹ for sEng) and rinsed 3 times with 1×PBST. The wells were blocked with 1% BSA. A dilution of target proteins at various concentrations in FBS were injected into the wells and the reaction was allowed to proceed at room temperature for 2 h. After rinsing the wells, 100 μl of detection antibody (60 ng μl⁻¹ for P1GF, 500 ng μl⁻¹ for sFIt-1, 50 ng μl⁻¹ for sEng) was added to the wells. The reaction was allowed to proceed at room temperature for 2 h. After rinsing 3 times, streptavidin-HRP was added to the wells. The reaction was allowed to proceed at room temperature for 20 min. The plate was rinsed 3 times, 100 μl of a substrate solution was added, followed by reaction for 20 min. After injection of 50 μl of a stop solution, the optical density was measured using an ELISA reader.

### 3. Analysis and Evaluation

### 3.1. Synthesis of Encoded Hydrogel Particles

Referring to FIGS. 1 and 2, cylindrical encoded hydrogel particles with up to 8 gear-shaped projections were synthesized by stop flow lithography (SFL). After synthesis of the encoded hydrogel particles, thiolated antibodies were bound to unreacted ends in the form of carbon-carbon double bonds of the hydrogel particles through the thiol-ene reaction. As a result, the antibodies are loaded into the hydrogel particles (see FIG. 3). Binding of the antibodies during synthesis of the particles might induce aggregation of the antibodies due to immiscibility with the photoinitiator. However, binding of the antibodies in a protein stabilization solution after synthesis of the particles enables loading of the antibodies into the particles with high density. Therefore, loading of the antibodies into the synthesized hydrogel particles ensures higher analytical sensitivity than binding of the antibodies during synthesis of the particles.

### 3.2. Colorimetric Reactions

In this experimental example, color development was induced through an enzyme-substrate reaction in the hydrogel particles. To this end, alkaline phosphatase (ALP) and 5-bromo-4-chloro-3-indolyl phosphate/nitroblue tetrazolium (BCIP/NBT) were used as an enzyme and a substrate, respectively. For a colorimetric reaction in the hydrogel particles, an antigen was specifically bound to a capture antibody and a biotinylated secondary antibody. The biotinylated secondary antibody was bound to streptavidin-ALP. After that, a BCIP/NBT substrate solution was introduced. The BCIP/NBT substrate was converted into an insoluble colorimetric material through an enzyme-substrate reaction in the hydrogel by the enzyme ALP. The insoluble colorimetric material was accumulated and amplified. In this course, the hydrogel particles were colored dark purple (see FIGS. 4 and 5). The insoluble colorimetric material aggregated locally in a hydrophilic environment and was fixed to a network of the hydrogel particles.

### 3.3. Protein Single Detection

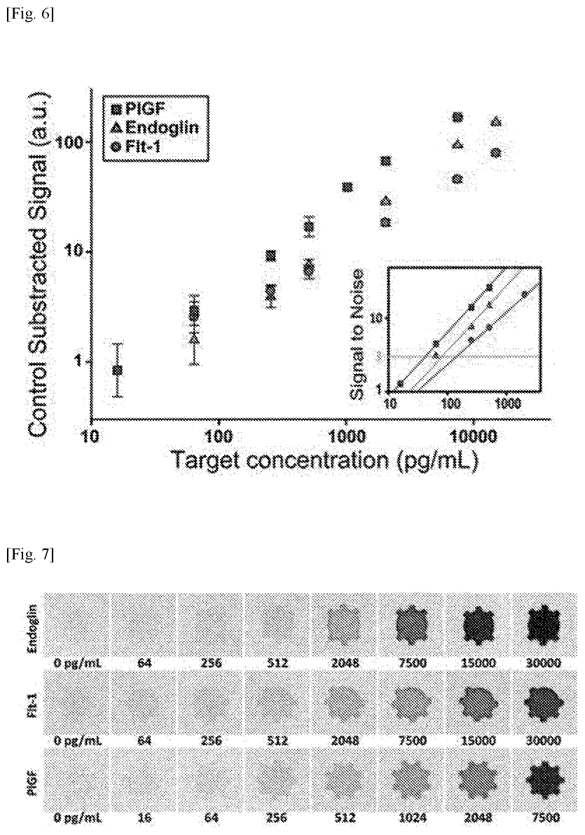

The detectable regions and minimum concentrations of three types of preeclampsia-related proteins (P1GF, Flt-1, and Endoglin) were investigated. The results are shown in FIGS. 6 and 7. FIG. 6 graphically shows the results of single detection of three types of preeclampsia-related proteins by a colorimetric detection method. FIG. 7 shows images revealing the results of single detection of three types of preeclampsia-related proteins by a colorimetric detection method.

As a result, the detectable regions of P1GF, Flt-1, and Endoglin were found to be 41.3-7500 pg μl⁻¹, 136.3-30000 pg μl⁻¹, and 73.5-15000 pg μl⁻¹, respectively, which exceed the results of ELISA obtained by measuring absorbance with a spectrometer (31.2-2000 pg μl⁻¹ for P1GF, 125-8000 pg μl⁻¹ for Flt-1, and 125-8000 pg μl⁻¹ for Endoglin).

### 3.4. Protein Multiplexed Detection

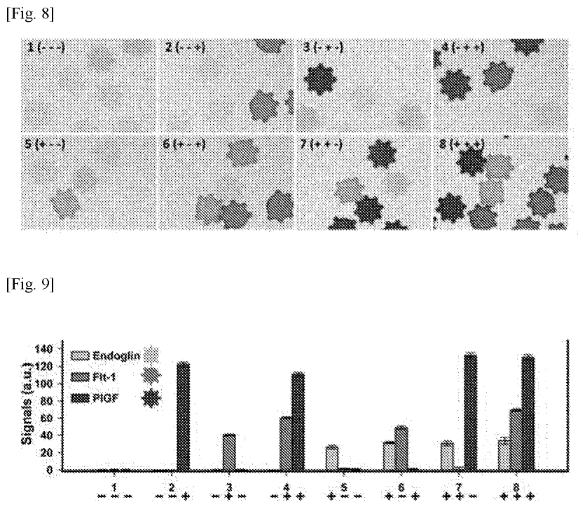

Multiplexed detection of three types of proteins (P1GF, Flt-1, and Endoglin) was performed. The results are shown in FIGS. 8 and 9. FIG. 8 shows images revealing the results of multiplexed detection of three types of preeclampsia-related proteins by a colorimetric detection method. FIG. 9 graphically shows the results of multiplexed detection of three types of preeclampsia-related proteins by a colorimetric detection method.

In the single detection as well as the multiplexed detection, the three types of proteins (P1GF, Flt-1, and Endoglin) did not show cross-reactivity in 8 cases depending on the presence or absence of each protein, The recovery rates were 92.6% for P1GF, 125.2% for Flt-1, and 122.9% for Endoglin, which were distributed within the generally acceptable range (70-130%).

### 3.5. Detection of Proteins in Plasma Samples

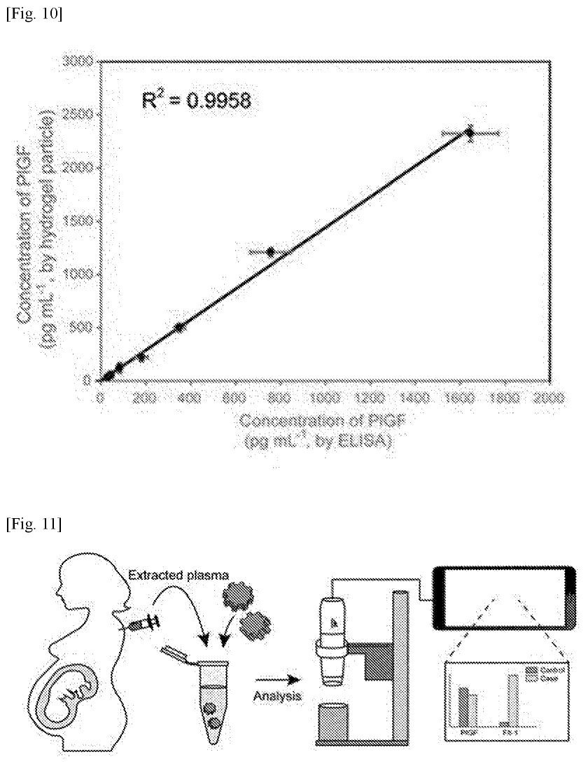

After different concentrations of P1GF were spiked in plasma samples from healthy subjects, P1GF was detected through ELISA, which is most commonly used for colorimetric reaction and protein detection. FIG. 10 compares the results of detection of proteins spiked in plasma by a colorimetric detection method with the results of ELISA.

Referring to FIG. 10, the colorimetric reaction and ELISA showed a linear relationship, indicating that the colorimetric reaction can be applied to the detection of proteins in real plasma samples.

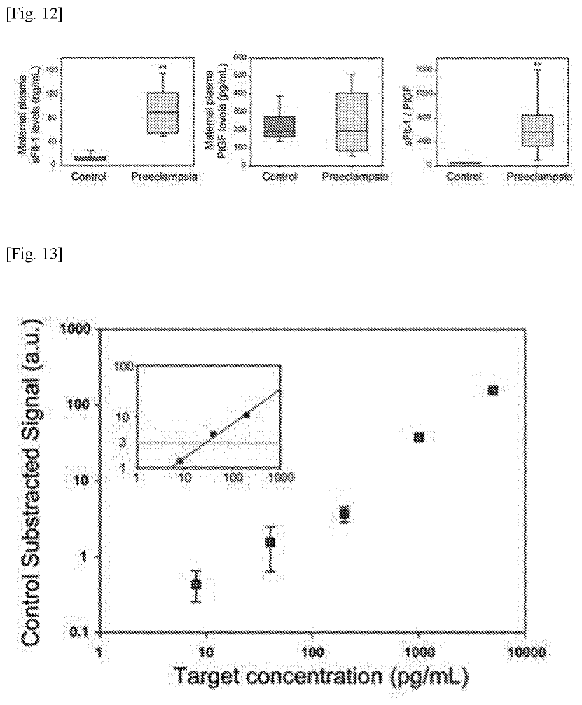

FIG. 11 is diagram schematically illustrating a process in which a colorimetric detection method is applied to plasma extracted from a real preeclampsia patient and a USB microscope and a smartphone are used for analysis. Based on the above results, multiplexed detection of P1GF and Flt-1 was performed in plasma samples from real preeclampsia patients and healthy subjects. The results are shown in FIG. 12. FIG. 12 shows the results of multiplexed detection of two types of proteins in plasma samples extracted from real preeclampsia patients and healthy subjects by a colorimetric detection method.

As a result, very different concentrations of P1GF and Flt-1 in the plasma samples made it difficult to perform multiplexed detection. The amount of Flt-1 and the Flt-1/P1GF ratio used to discriminate preeclampsia were significantly different between the patient and normal groups. However, there was no significant difference in the amount of P1GF, which is believed to be because the numbers of cases in the patient and normal groups were small (normal: 5 cases, patient: 5 cases).

### 3.6. Nucleic Acid Detection

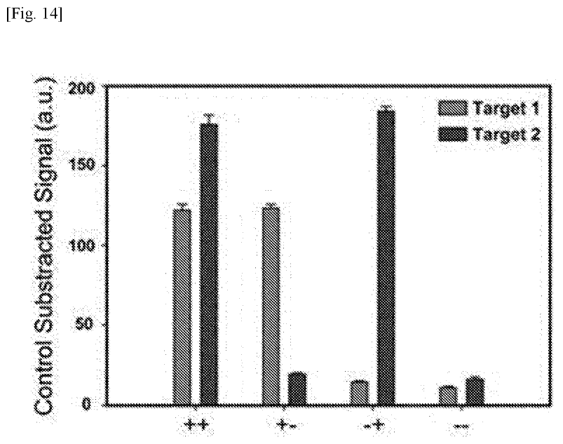

Nucleic acids were detected using colorimetric reactions. The results are shown in FIGS. 13 and 14. FIG. 13 shows the results of single detection of nucleic acids by a colorimetric detection method. FIG. 14 shows the results of multiplexed detection of nucleic acids by a colorimetric detection method. The detectable LoD of the detection for nucleic acids through colorimetric reactions was found to be 33.72 amol (see FIG. 13). High specificity was also observed in a cross-reactivity test using two types of targets (see FIG. 14).

Although the present invention has been described herein with reference to the specific embodiments, these embodiments do not serve to limit the invention and are set forth for illustrative purposes. It will be apparent to those skilled in the art that modifications and improvements can be made without departing from the spirit and scope of the invention.

Such simple modifications and improvements of the present invention belong to the scope of the present invention, and the specific scope of the present invention will be clearly defined by the appended claims.

### INDUSTRIAL APPLICABILITY

According to the present invention, target analytes specifically bind to probes loaded into hydrogel particles and an insoluble colorimetric material is accumulated and amplified in the hydrogel particles to obtain results comparable to the detection sensitivity and specificity achieved using a fluorescent material even without using expensive analysis systems or a separate space. Therefore, the present invention is considered industrially applicable.

1. A method for colorimetric detection of target analytes based on hydrogel particles comprising (a) reacting a sample containing target analytes with hydrogel particles loaded with probes specifically binding to the target analytes and (b) accumulating and amplifying an insoluble colorimetric material in the hydrogel particles to label the target analytes bound to the probes wherein the hydrogel particles form a polymer network, the probes are bound to and loaded into the polymer network, and the insoluble colorimetric material is fixed to the polymer network.

2. The method according to claim 1, wherein the hydrogel particles are geometrically shaped and encoded to identify the probes.

3. The method according to claim 1, wherein the probes are loaded during or after synthesis of the hydrogel particles.

4. The method according to claim 3, wherein each of the probes loaded after synthesis of the hydrogel particles comprises a capture portion specifically binding to the corresponding target analyte and a functional group connected to the capture portion and bonded to an unreacted end in the form of a carbon-carbon double bond connected to the polymer network.

5. The method according to claim 1, wherein the probes are compounds, oligonucleotides, oligosaccharides, proteins, antibodies, peptides or aptamers that specifically bind to the target analytes.

6. The method according to claim 4, wherein the functional groups are selected from the group consisting of thiol (—SH) and amine groups (—NH₂).

7. The method according to claim 1, wherein step (b) comprises conjugating an enzyme to the target analytes bound to the probes and adding a substrate reacting with the enzyme to produce the insoluble colorimetric material.

8. The method according to claim 7, wherein the conjugation of the enzyme comprises adding secondary binding materials specifically binding to the target analytes and adding the enzyme for binding to the secondary binding materials.

9. The method according to claim 8, wherein the secondary binding materials are compounds, oligonucleotides, oligosaccharides, proteins, antibodies, peptides or aptamers that specifically bind to the target analytes.

10. The method according to claim 7, wherein enzyme is selected from alkaline phosphatase (ALP), β-galactosidase, peroxidase, luciferase, cytochrome P450, and combinations thereof.

11. The method according to claim 7, wherein the substrate is selected from bromochloroindolyl phosphate (BCIP)/nitroblue tetrazolium (NBT), naphthol-AS-B1-phosphate, p-nitrophenyl phosphate (PNPP), enhanced chemifluorescence (ECF), 4-chloronaphthol, 3,3′-diaminobenzidine (DAB), 3-amino-9-ethylcarbazole (AEC), 3,3′,5,5′-tetramethylbenzidine (TMB), 4-chloronaphthol, 3,3′-diaminobenzidine (DAB), 3-amino-9-ethylcarbazole (AEC), 6-chloro-3-indolyl-o-D-galactopyranoside (Red-gal), and combinations thereof.

12. The method according to claim 1, wherein the target analytes comprise one or more materials selected from the group consisting of DNA, RNA, proteins, exosomes, and viruses.
